(19) United States
(12) Plant Patent Application Publication
(10) Pub. No.: US 2009/0172854 P1
(43) Pub. Date: Jul. 2, 2009

(54) Chrysanthemum named 'Zanmuflamin

(50) ;

(75) Inventor: Wilhelmus Bernardus Blom, Leimuiden (NL)

Correspondence Address: STEPTOE & JOHNSON LLP, 1330 CONNECTICUT AVENUE, N.W., WASHINGTON, DC, 20036, US

(73) Assignee: Chrysanthemum Breeders Association Research B.V., Valkenburg Z-H (NL)

(21) Appl. No.: 12/003,596

(22) Filed: Dec. 28, 2007

(51) Int. Cl. A01H 5/00

(52) U.S. Cl. PLT/292

(57) ABSTRACT

A chrysanthemum plant named ‘Zanmuflamin’ characterized by its medium sized double type blooms with bright purple ray florets and prolific branching; natural season flower date September 11 (week 37); blooming for a period of 5 weeks.

### BACKGROUND OF THE INVENTION

‘Zanmuflamin’ is a product of a breeding and selection program for outdoor pot mums (garden mums) which had the objective of creating new chrysanthemum cultivars with a double type flower, a natural season flower date around September 10 (week 37); blooming for a period of 5 weeks. The new plant of the present invention comprises a new and distinct cultivar of chrysanthemum plant ‘Zanmuflamin’ is a seedling resulting from a crossing of the plants id 16.609×id 871. The new and distinct cultivar was discovered and selected as a flowering plant by Wilhelmus Bernardus Blom on a cultivated field in Rijsenhout, The Netherlands in 2004. The first act of asexual production of ‘Zanmuflamin’ was accomplished when vegetative cuttings were taken from the initial selection in 2004 and propagated further in a controlled environment in Rijsenhout, The Netherlands. The new cultivar has been found to retain its distinctive characteristics through successive propagations.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

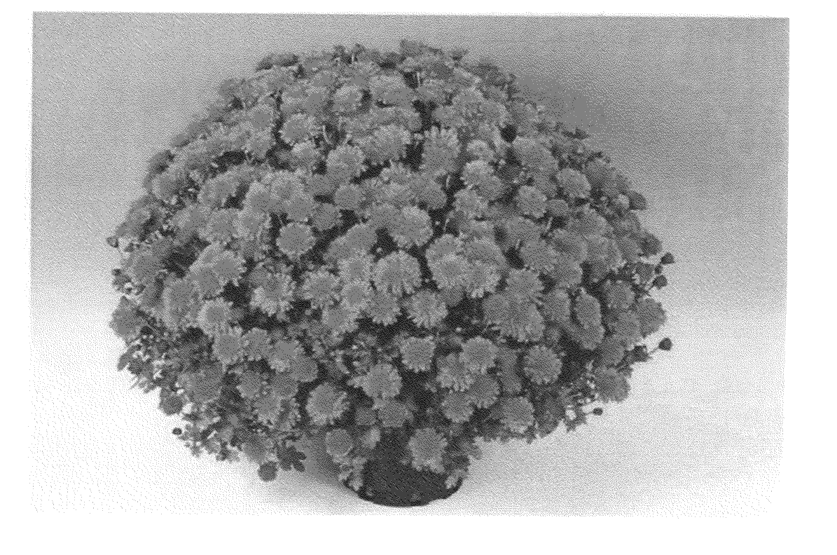

FIG. 2 shows the various stages of bloom of the new cultivar.

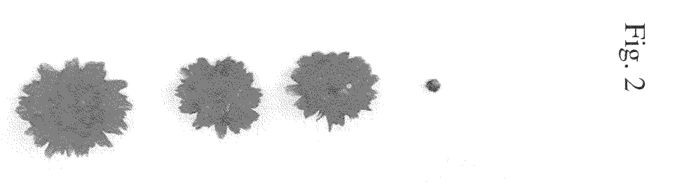

FIG. 3 shows the various stages of foliage of the new cultivar.

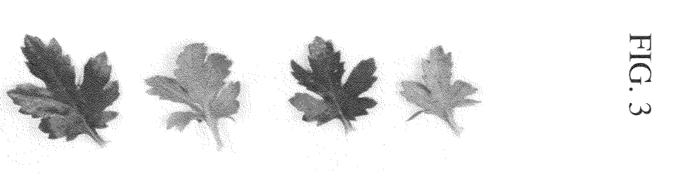

### DESCRIPTION OF THE INVENTION

The observations and measurements were gathered from plants grown out door in Rijsenhout, The Netherlands under natural day length and temperature and planted in week 23 in 2006. The natural blooming date of this crop was September 11 (week 37). The average height of the plants was 30 cm. No growth retardants were used. No tests were done on disease or insect resistance or susceptibility. No tests were done on cold or drought tolerance. This new variety produces medium sized double type blooms with bright purple ray florets blooming for a period of 5 weeks.

From the cultivars known to inventor the most similar existing cultivar in comparison to ‘Zanmuflamin’ is ‘Venus purple (U.S. Plant Pat. No. 14,378). When ‘Venus purple and ‘Zanmuflamin’ are being compared the following difference is noticed: The difference of ‘Venus purple and ‘Zanmuflamin’ are (1) flower colour and (2) natural season blooming date. (1) The flower colour of ‘Zamuflamin’ is more pink than of ‘Venus Purple’ (2) ‘Venus purple’ flowers later than ‘Zanmuflamin’

The following is a description of the plant and characteristics that distinguish ‘Zanmuflamin’ as a new and distinct variety.

The color designations are taken from the plant itself. Accordingly, any discrepancies between the color designations and the colors depicted in the photographs are due to photographic tolerances. The color chart used in this description is: The Royal Horticultural Society Colour Chart, edition 2001.

TABLE 1
Botanical Description of chrysanthemum plant ‘Zanmuflamin’

| | |
|---|---|
| Bud | |
| Size | Small ;cross-section 1 cm, height 0.8 cm |
| Outside Color | Greyed-purple 186D |
| Phyllaries | 2 rows |
| Phyllaries among disc- | Not present |
| florets | |
| Bloom | |
| Type | Double |
| Height | 1.5 cm |
| Size | 4.5 cm |
| Phyllary number | 12-14 |
| Phyllary color | Green 138A |
| Phyllary length and | 2 cm and 2 mm |
| width | |
| Peduncle length | 4-4.5 cm |
| Peduncle color | Green 138B |
| Number of blooms per | Approx. 5 blooms per branch |
| branch | |
| Performance on the | 5 weeks |
| plant | |
| Seeds | Produced in small quantities, ovate grey-brown |
| | 199A, 1½ mm in length. |
| Fragrance | Typical chrysanthemum, slightly |
| Color | |
| Center of the flower | Immature stage: Red-purple 58A |
| | Mature stage: Red-purple 58A |
| Color of upper surface | Red-purple 59D |
| of the ray-florets | |
| Color of the lower | Greyed-purple 186C (at top) to 186D (at base) |
| surface of the ray- | |
| florets | |
| Tonality from Distance | A garden mum with bright purple flowers |
| Color of the ray-florets | Greyed-purple 186B |
| after aging of the plant | |
| Ray florets | |
| Texture | Upper and under side smooth |
| Number | 200-220 |
| Cross-section | Flat |
| Longitudinal axis off | Straight |
| majority | |
| Length of corolla tube | 0.3-0.4 cm |
| Ray-floret margin | Entire |
| Ray-floret length | 2-2.2 cm |
| Ray-floret width | 0.3-0.5 cm |
| Ratio length/width | High |
| Shape of tip | Rounded |
| Disc florets | Absent |
| Receptacle sshape | Conical raised |
| Reproductive Organs | |
| Stamen | Lacking |
| Styles | Short |
| Style color | Yellow 13A |
| Style Length | 3 mm |
| Stigma color | Yellow-green 144A |
| Stigma Width | 1 mm |
| Ovaries | Enclosed in clayx |
| Plant | |
| Form | Grown as a spray type potmum, outdoor mounded |
| | and round |
| Growth habit | Spherical shape |
| Growth rate | Medium vigour |
| Height | 30 cm |
| Width | 40 cm |
| Stem Color | Greyed-brown 199C |
| Stem Strength | Strong |
| Stem Anthocyanin | Present |
| Coloration | |
| Internode length | 1-2 cm |
| Length of lateral | From top to bottom 15-18 cm |
| branch | |
| Lateral branch color | Yellow-green 146C |
| Lateral branch, | Medium strength |
| attachment | |
| Branching (average | Good with 6-7 breaks after pinching |
| number of lateral | |
| branches) | |
| Natural season | September 11 (week 37) |
| blooming date | |
| Foliage | |
| Leaf color | Upper side Green 143b |
| | Lower side Green 143C |
| Color midvein | Upper side Yelloww-green 147D |
| | Lower side Yellow-green 148D |
| Size | Small.; length 4.5-6.5 cm, width 3-3.5 cm |
| Quantity (number per | 16 |
| lateral branch) | |
| Shape | Elliptic |
| Texture upper side | Glabrous |
| Texture under size | Pubescent |
| Venation arrangement | Palmate |
| Shape of the margin | Serrated |
| Shape of Base of Sinus | Acute |
| Between Lateral Lobes | |
| Margin of Sinus | Diverging |
| Between Lateral Lobes | |
| Shape of Base | Truncate |
| Apex | Mucronulate |
| Petiole length | 1 cm |
| Petiole color | Yellow-green 147D |

TABLE 2
Differences with the comparison variety (grown under identical
conditions)

| | ‘Zanmuflamin’ | ‘Venus purple’ |
|---|---|---|
| Flower colour | Red-purple 59D | Red-purple 60A |
| Natural season | September 11 (week 37) | September 18 (week 38) |
| blooming date | | |

I claim:

1. A new and distinct variety of chrysanthemum plant as described and illustrated.
